(19) United States
(12) Patent Application Publication
(10) Pub. No.: US 2012/0157793 A1
(43) Pub. Date: Jun. 21, 2012

(54) MEDICATION INTAKE ANALYZER

(75) Inventor: Mary Louise MacDonald, Boston, MA (US)

(73) Assignee: General Electric Company, Schenectady, NY (US)

(21) Appl. No.: 12/973,409

(22) Filed: Dec. 20, 2010

(51) Int. Cl. A61B 5/00; G06Q 50/00

(52) U.S. Cl. 600/301; 705/3

(57) ABSTRACT

An example method for medication intake analysis and reporting. The method includes obtaining a medication order including identification of a medication and a property associated with the medication, where the medication order is associated with a patient. The method includes obtaining a value representing a characteristic of a physical state of the patient, where the value is measured by a sensor in contact with the patient. The method includes determining whether the patient has taken the medication associated with the medication order based on a comparison of the value representing a characteristic of the physical state of the patient. The method includes reporting whether the patient has taken the medication associated with the medication order via a user interface.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND

Healthcare environments, such as hospitals or clinics, include information systems, such as hospital information systems (HIS), radiology information systems (RIS), clinical information systems (CIS), and cardiovascular information systems (CVIS), and storage systems, such as picture archiving and communication systems (PACS), library information systems (LIS), and electronic medical records (EMR). Information stored may include patient medication orders, medical histories, imaging data, test results, diagnosis information, management information, and/or scheduling information, for example.

Healthcare providers may desire to monitor patient medication intake based on the patient medication orders stored in healthcare information systems. Sensors, such as sweat sensors and heart rate sensors, can measure certain characteristics of a physical state of a person in contact with the sensors. In the healthcare environment, the values measured by the sensors can be used to analyze the medication that may have been ingested by a patient in contact with the sensors. This analysis is based on specific properties that are associated with medications, such as dosage, concentration, and/or chemical composition.

### BRIEF SUMMARY

Certain examples provide methods, apparatus, and articles of manufacture for medication intake analysis and reporting.

Certain examples provide a method for medication intake analysis and reporting. The method includes obtaining a medication order including identification of a medication and a property associated with the medication, where the medication order is associated with a patient. The method includes obtaining a value representing a characteristic of a physical state of the patient, where the value is measured by a sensor in contact with the patient. The method includes determining whether the patient has taken the medication associated with the medication order based on a comparison of the value representing a characteristic of the physical state of the patient. The method includes reporting whether the patient has taken the medication associated with the medication order via a user interface.

Certain examples provide a medication intake analysis and reporting system. The system includes a medication analyzer to obtain a medication order including identification of a medication and a property associated with the medication, where the medication order is associated with a patient. The system includes a patient analyzer to obtain a value representing a characteristic of a physical state of the patient, where the value is measured by a sensor in contact with the patient. The patient analyzer is to determine whether the patient has taken the medication associated with the medication order based on a comparison of the value representing a characteristic of the physical state of the patient with the property associated with the medication. The system includes a report generator to report whether the patient has taken the medication associated with the medication order.

Certain examples provide a tangible computer readable medium having a set of instructions for execution on a processing device, the set of instructions implementing a method for medication intake analysis and reporting. The method includes obtaining a medication order including identification of a medication and a property associated with the medication, where the medication order is associated with a patient. The method includes obtaining a value representing a characteristic of a physical state of the patient, where the value is measured by a sensor in contact with the patient. The method includes determining whether the patient has taken the medication associated with the medication order based on a comparison of the value representing a characteristic of the physical state of the patient. The method includes reporting whether the patient has taken the medication associated with the medication order via a user interface.

### BRIEF DESCRIPTION OF SEVERAL VIEWS OF THE DRAWINGS

FIG. 1 illustrates a block diagram of an example healthcare system.

FIGS. 2a and 2b illustrate an example implementation of the example medication intake analyzer of FIG. 1.

FIG. 3 illustrates an example medication intake analyzer of FIG. 1.

FIG. 4 illustrates a flow diagram for an example method of medication intake analysis and reporting.

FIG. 5 shows a block diagram of an example processor system that may be used to implement systems and methods described herein.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF CERTAIN EXAMPLES

Although the following discloses example methods, systems, articles of manufacture, and apparatus including, among other components, software executed on hardware, it should be noted that such methods and apparatus are merely illustrative and should not be considered as limiting. For example, it is contemplated that any or all of these hardware and software components could be embodied exclusively in hardware, exclusively in software, exclusively in firmware, or in any combination of hardware, software, and/or firmware. Accordingly, while the following describes example methods, systems, articles of manufacture, and apparatus, the examples provided are not the only way to implement such methods, systems, articles of manufacture, and apparatus.

When any of the appended claims are read to cover a purely software and/or firmware implementation, in an embodiment, at least one of the elements is hereby expressly defined to include a tangible medium such as a memory, DVD, CD, Blu-ray, etc., storing the software and/or firmware.

In certain examples, a medication order associated with a patient is stored in a healthcare system. The medication order includes a medication that has been prescribed to the patient by a healthcare provider. Additionally, the medication order includes various properties associated with the prescribed medication, such as recommended dosage, concentration, or chemical composition.

In certain examples, the healthcare system includes a medication intake analyzer. The medication intake analyzer is connected to a sensor, such as a fiber optic sweat sensor or heart rate sensor, to measure certain characteristics of a physical state of the patient in contact with the sensor. The medication intake analyzer uses the sensor data and the medication order stored in the healthcare system to determine whether the patient has taken the prescribed medication detailed in the medication order. Thus, the medication intake analyzer and sensors can be customized to analyze a wide range of medications needed to treat various medical conditions.

In certain examples, the medication intake analyzer is connected to a user interface to inform the patient of whether the patient has taken the prescribed medication detailed in the medication order. The user interface is implemented using a display and/or audio feedback to allow the patient to see and/or hear the results of the medication intake analysis. This implementation provides patient feedback based on the needs of that particular patient. For example, the user interface may display the results of the medication intake analysis when the patient has suffered hearing loss. Alternatively, the user interface may provide the results of the medication intake analysis via audio feedback when the patient has diminished eyesight.

In certain examples, the medication intake analyzer sends the results of the medication intake analysis to the healthcare system to maintain a record of whether the patient has taken the prescribed medication from the medication order. Maintaining this record allows the prescribing healthcare provider or any other healthcare provider to monitor the patient's medication intake via the healthcare system.

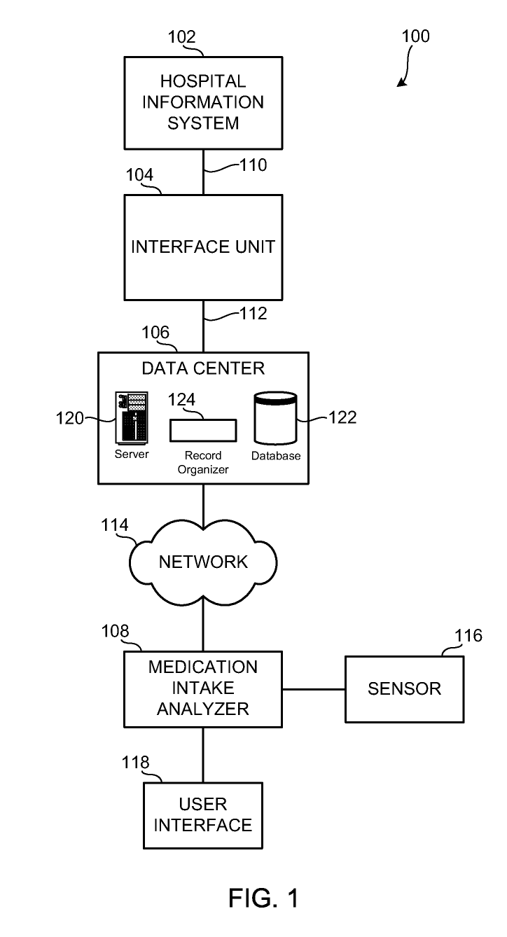

FIG. 1 shows a block diagram of an example healthcare system 100 capable of implementing the example methods and systems described herein. The example healthcare system 100 includes a hospital information system (HIS) 102, an interface unit 104, a data center 106, and a medication intake analyzer 108. In the illustrated example, the HIS 102 is housed in a healthcare facility and locally archived. However, in other implementations, the HIS 102 can be housed in one or more other suitable locations. In certain implementations, the HIS 102 can be implemented remotely via a thin client and/or downloadable software solution. Information (e.g., medication orders, scheduling, test results, observations, diagnosis, etc.) can be entered into the HIS 102 by healthcare providers (e.g., radiologists, physicians, and/or technicians) before and/or after patient examination.

The HIS 102 stores medical information such as medication orders, clinical reports, patient information, and/or administrative information received from, for example, personnel at a hospital, clinic, and/or a physician's office.

The interface unit 104 includes a hospital information system interface connection 110 and a data center interface connection 112. The interface unit 104 facilities communication between the HIS 102 and the data center 106. The interface connection 110 can be implemented by, for example, a Wide Area Network (“WAN”) such as a private network or the Internet. Accordingly, the interface unit 104 includes one or more communication components such as, for example, an Ethernet device, an asynchronous transfer mode (“ATM”) device, an 802.11 device, a DSL modem, a cable modem, a cellular modem, etc. In turn, the data center 106 communicates with the medication intake analyzer 108 via a network 114. The medication intake analyzer 108 may be implemented at a plurality of locations (e.g., a hospital, clinic, doctor's office, other medical office, assisted living facility, or terminal, etc.). The network 114 is implemented by, for example, the Internet, an intranet, a private network, a wired or wireless Local Area Network, and/or a wired or wireless Wide Area Network.

The example data center 106 of FIG. 1 is an archive to store information such as, for example, medication orders, data, medical reports, and/or, more generally, patient medical records. In addition, the data center 106 can also serve as a central conduit to information located at other sources such as, for example, local archives, hospital information systems (e.g., the HIS 102), radiology information systems, or medical imaging/storage systems. That is, the data center 106 can store links or indicators (e.g., identification numbers, patient names, or record numbers) to information. In the illustrated example, the data center 106 is managed by an application server provider (“ASP”) and is located in a centralized location that can be accessed by a plurality of systems and facilities (e.g., hospitals, clinics, doctor's offices, other medical offices, and/or terminals). In some examples, the data center 106 can be spatially distant from the HIS 102 (e.g., at General Electric® headquarters).

The example data center 106 of FIG. 1 includes a server 120, a database 122, and a record organizer 124. The server 120 receives, processes, and conveys information to and from the components of the healthcare system 100. The database 122 stores the medical information described herein and provides access thereto. The example record organizer 124 of FIG. 1 manages patient medical histories, for example. The record organizer 124 can also assist in procedure scheduling, for example.

In operation, the interface unit 104 receives medication orders, medical reports, administrative information, and/or other clinical information from the HIS 102 via the interface connection 110. If necessary (e.g., when different formats of the received information are incompatible), the interface unit 110 translates or reformats (e.g., into Structured Query Language (“SQL”) or standard text) the medical information, such as medication orders, to be properly stored at the data center 106. The reformatted medical information can be transmitted using a transmission protocol to enable different medical information to share common identification elements, such as a patient name or social security number. Next, the interface unit 104 transmits the medical information to the data center 106 via the data center interface connection 112. Finally, medical information is stored in the data center 106.

The medical information, such as a medication order, is later retrievable at the medication intake analyzer 108 (e.g., by its common identification element, such as a patient name or record number). The medication intake analyzer 108 can be any equipment (e.g., a personal computer) capable of executing software that permits electronic data (e.g., medication orders) to be acquired, stored, or transmitted for operation. As shown in FIG. 1, the medication intake analyzer 108 is connected to the network 114 and, thus, can communicate with the data center 106, and/or any other device coupled to the network 114.

The medication intake analyzer 108 receives input from a patient via a sensor 116, such as, for example, a fiber optic sweat sensor, heart rate sensor, etc. The medication intake analyzer 108 implements a user interface 118 to enable a patient to interact with the healthcare system 100. For example, the patient comes in contact with the sensor 116 and, in response to the medication intake analysis based on a medication order retrieved from the healthcare system 100, the user interface 118 reports to the patient, via a visual display and/or audio feedback, whether the patient has taken his or her prescribed medication. The medication intake analyzer 108 and sensor 116 may be customized to analyze a wide range of medications needed to treat various medical conditions.

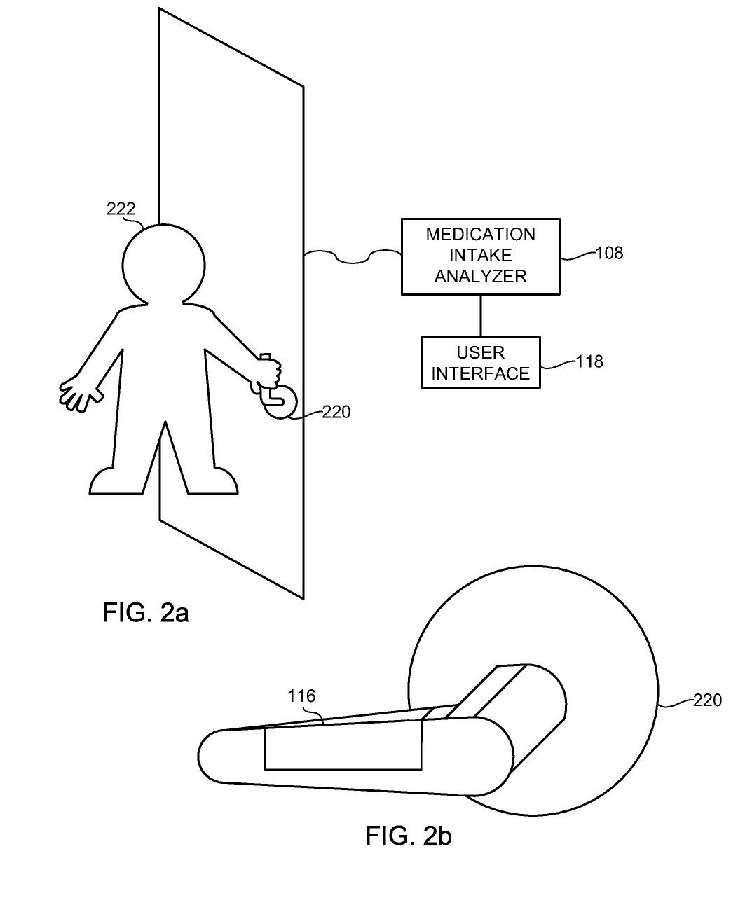

FIGS. 2a and 2b illustrate an example implementation of an example medication intake analyzer 108 of FIG. 1. The example medication intake analyzer 108 may be implemented at a plurality of locations (e.g., a hospital, clinic, doctor's office, other medical office, assisted living facility, or terminal, etc.). In this example, the example sensor 116 of FIG. 1 is placed on a door handle 220 at an assisted living facility. However, the sensor 116 may be placed on any surface that allows for patient contact, such as, for example, a medicine cabinet or a hospital bed guard rail. The sensor 116 may be a fiber optic sweat sensor or heart rate sensor, or any other sensor capable of measuring a characteristic of a patient's physical state. Additionally, a component may be added to the sensor and/or door handle to confirm that a specific patient's medication intake is being analyzed. For example, a patient can be identified based on one or more of a patient identification card, an electronic record associating the patient with a specific room, a radio frequency identification (RFID) on and/or in the patient that can be scanned, a near field communication device on the patient, and/or other identifier that can be detected and matched with a particular patient. Identifying the patient helps ensure that the correct sensor data and medication information is being used in the medication intake analysis. In one example, a patient identification card can be scanned by the component and the patient identification information can be used by the example medication intake analyzer 108 to ensure that the proper medication order is being used in the analysis.

In this example implementation, a patient 222 opens a door of an assisted living facility using the door handle 120 and, thus, the patient 222 comes in contact with the sensor 116 located on the door handle 220. In this example, the sensor 116 measures the sweat and heart rate of the patient 222. The sensor 116 sends the measured data to the medication intake analyzer 108. The medication intake analyzer 108 determines whether the patient 222 has taken his medication. The medication intake analyzer 108 then reports whether the patient 222 has taken his medication to the patient 222 via the user interface 118 of FIG. 1. The user interface 118 may be implemented using a display and/or audio feedback.

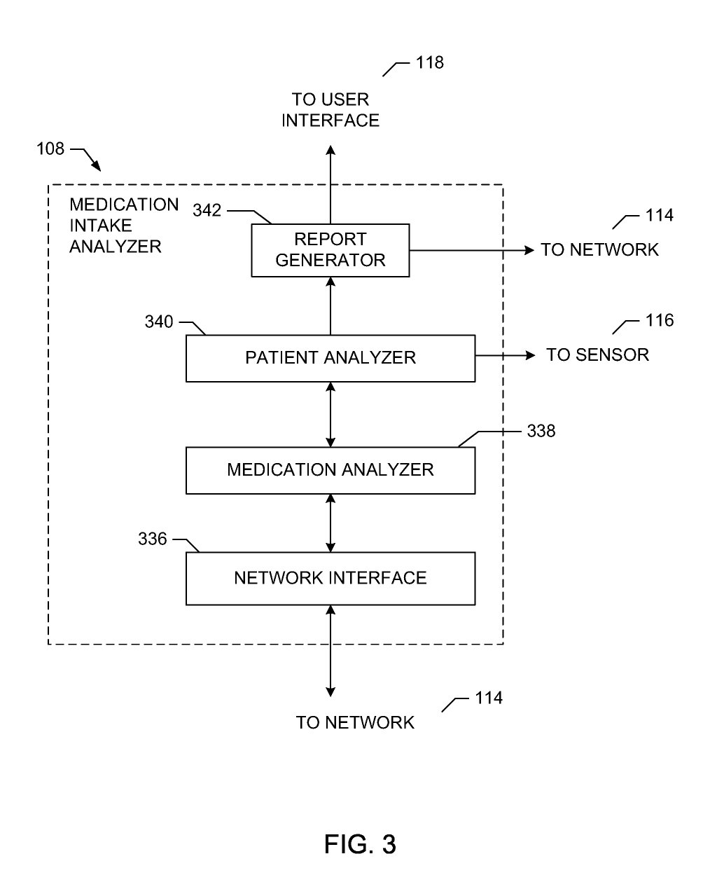

FIG. 3 illustrates an example medication intake analyzer 108 of FIG. 1. The medication intake analyzer 108 includes a network interface 336, a medication analyzer 338, a patient analyzer 340 and a report generator 342. The network interface 336 interfaces with the network 114 of FIG. 1 to obtain a medication order associated with a patient from the healthcare system 100 of FIG. 1. Via the network interface 336, the medication analyzer 338 obtains the medication order. The medication order is to include an identification of a medication and a property associated with the medication, such as, for example, a dosage, concentration, or chemical composition. The medication analyzer 338 identifies the medication property relevant to the analysis based on a characteristic of a physical state of a patient that will be measured by the sensor 116 of FIG. 1.

The patient analyzer 340 obtains a value representing a characteristic of a physical state of the patient. The value is measured by a sensor 116 of FIG. 1 that is in contact with the patient. The sensor 116 may be a fiber optic sweat sensor, a heart rate sensor, or any other sensor capable of measuring a characteristic of a physical state of a patient. Once the value measured by the sensor 116 has been obtained by the patient analyzer 340, the patient analyzer 340 determines whether the patient has taken the medication associated with the medication order. To make this determination, the patient analyzer 340 compares the value that represents the characteristic of the physical state of the patient measured by the sensor 116 to the property associated with the medication identified by the medication analyzer 338.

The report generator 342 generates a report that includes information regarding whether the patient has taken the medication associated with the medication order. The report generated by the report generator is reviewed by the patient via the user interface 118. The user interface 118 is presented to the patient using a display and/or audio feedback. Additionally or alternatively, the report generated by the report generator 342 is stored in the patient records in the healthcare system 100 of FIG. 1 via the network 114. Recording the medication intake in the patient records allows a prescribing healthcare provider or any other healthcare provider to monitor the medication intake of the patient.

The network interface 336, medication analyzer 338, patient analyzer 340 and report generator 342 can be implemented in software, hardware, firmware, and/or a combination of these elements. The network interface 336, medication analyzer 338, patient analyzer 340 and report generator 342 can be implemented separately and/or combined in various forms. The network interface 336, medication analyzer 338, patient analyzer 340 and report generator 342 can be implemented as a set of instructions/routines forming machine executable code stored on a machine accessible medium for execution by a computing/processing device, for example.

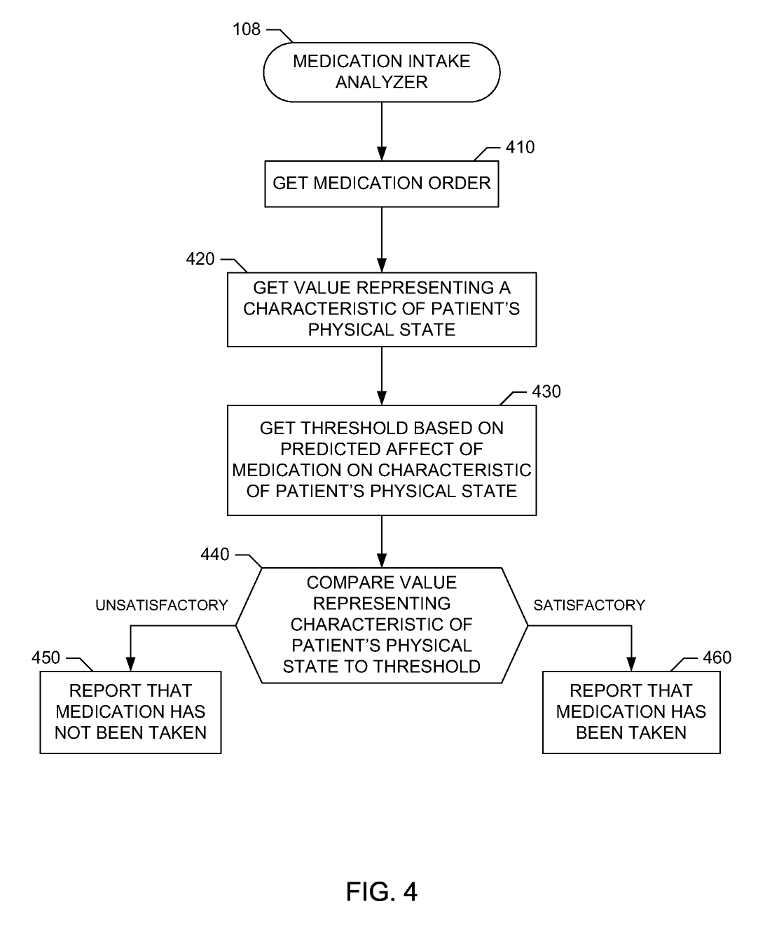

FIG. 4 illustrates a flow diagram for an example method of medication intake analysis and reporting. The example process(es) of FIG. 4 can be performed using a processor, a controller and/or any other suitable processing device. For example, the example process(es) of FIG. 4 can be implemented using coded instructions (e.g., computer readable instructions) stored on a tangible computer readable medium such as a flash memory, a read-only memory (ROM), and/or a random-access memory (RAM). As used herein, the term tangible computer readable medium is expressly defined to include any type of computer readable storage and to exclude propagating signals. Additionally or alternatively, the example process(es) of FIG. 4 can be implemented using coded instructions (e.g., computer readable instructions) stored on a non-transitory computer readable medium such as a flash memory, a read-only memory (ROM), a random-access memory (RAM), a cache, or any other storage media in which information is stored for any duration (e.g., for extended time periods, permanently, brief instances, for temporarily buffering, and/or for caching of the information). As used herein, the term non-transitory computer readable medium is expressly defined to include any type of computer readable medium and to exclude propagating signals.

Alternatively, some or all of the example process(es) of FIG. 4 can be implemented using any combination(s) of application specific integrated circuit(s) (ASIC(s)), programmable logic device(s) (PLD(s)), field programmable logic device(s) (FPLD(s)), discrete logic, hardware, firmware, etc. Also, some or all of the example process(es) of FIG. 4 can be implemented manually or as any combination(s) of any of the foregoing techniques, for example, any combination of firmware, software, discrete logic and/or hardware. Further, although the example process(es) of FIG. 4 are described with reference to the flow diagram of FIG. 4, other methods of implementing the process(es) of FIG. 4 can be employed. For example, the order of execution of the blocks can be changed, and/or some of the blocks described can be changed, eliminated, sub-divided, or combined. Additionally, any or all of the example process(es) of FIG. 4 can be performed sequentially and/or in parallel by, for example, separate processing threads, processors, devices, discrete logic, circuits, etc.

FIG. 4 illustrates a flow diagram for an example method 400 to implement the example medication intake analyzer 108 of FIGS. 1-3. At block 410, a medication order associated with a patient is obtained from a healthcare system. The medication order is to include an identification of a medication and a property associated with the medication, such as, for example, a dosage, concentration, and/or chemical composition.

At block 420, a value representing a characteristic of a physical state of the patient is obtained via a sensor that is in contact with the patient. The sensor may be a fiber optic sweat sensor, a heart rate sensor, and/or any other sensor that is capable of measuring a characteristic of a physical state of a patient.

At block 430, a threshold value is obtained based on the property associated with the medication in the medication order. The threshold value is calculated based on a predicted affect of the property associated with the medication on the measured value representing the characteristic of the physical state of the patient. For example, where a specific medication is to be taken, a threshold value can be calculated that predicts the value that will be collected by a sweat sensor.

At block 440, it is determined whether the patient has taken the medication associated with the medication order. Whether the patient has taken the medication associated with the medication order is determined by comparing the value representing the characteristic of the physical state of the patient to the determined threshold value. This comparison can result in a satisfactory or an unsatisfactory result to indicate whether or not the patient has taken the medication. For example, if a sensor is used to collect the heart rate of a patient, this value is compared to a threshold calculated based on the predicted effect of the prescribed medication on the patient's heart rate. In this example, if the patient's heart rate is above this calculated threshold, the result is unsatisfactory and indicates that the patient has not taken the prescribed medication. In another example, if a sensor is used to collect the sweat composition of a patient, this value is compared to a threshold calculated based on the predicted effect of the prescribed medication on the patient's sweat composition. In this example, if the patient's sweat composition concentration is below this calculated threshold, the result is satisfactory and indicates that the patient had taken the prescribed medication.

If the value representing the characteristic of the physical state of the patient is determined to be unsatisfactory, at block 450, it is to be reported that the medication associated with the medication order has not been taken. If the value representing the characteristic of the physical state of the patient is determined to be satisfactory, at block 460, it is to be reported that the medication associated with the medication order has been taken. The report that the medication associated with the medication order has or has not been taken may be reported to the patient, via, for example, a display and/or audio feedback, and/or may be reported to the healthcare system for monitoring by a healthcare provider.

For example, if the value representing the characteristic of the physical state of the patient is greater than the determined threshold value, at block 450, it is to be reported that the medication associated with the medication order has not been taken. If the value representing the characteristic of the physical state of the patient is less than the determined threshold value, at block 460, it is to be reported that the medication associated with the medication order has been taken, for example. Alternatively and/or in addition, a value less than the determined threshold value may indicate that the medication has not been taken; while a value greater than the determined threshold value may indicate that the medication has been taken. The report that the medication associated with the medication order has or has not been taken may be reported to the patient via, for example, a display and/or audio feedback, and/or may be reported to the healthcare system for monitoring by a healthcare provider.

One or more of the blocks of the method 400 can be implemented alone or in combination in hardware, firmware, and/or as a set of instructions in software, for example. Certain examples can be provided as a set of instructions residing on a computer-readable medium, such as a memory, hard disk, DVD, or CD, for execution on a general purpose computer or other processing device.

Certain examples can omit one or more of these blocks and/or perform the blocks in a different order than the order listed. For example, some steps may not be performed in certain examples. As a further example, certain steps can be performed in a different temporal order, including simultaneously, than listed above.

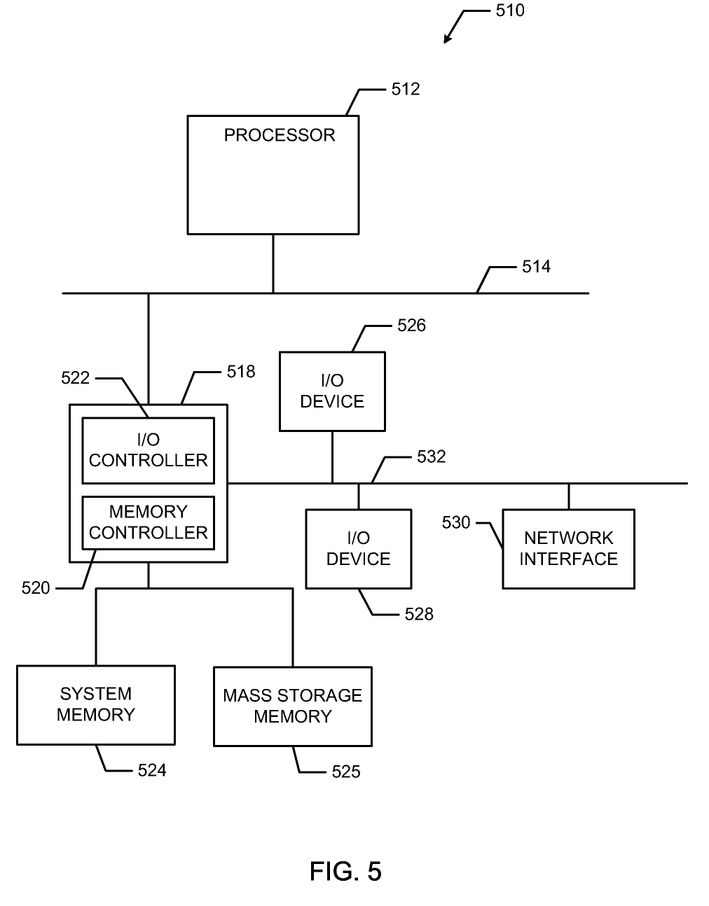

FIG. 5 is a block diagram of an example processor system 510 that can be used to implement systems and methods described herein. As shown in FIG. 5, the processor system 510 includes a processor 512 that is coupled to an interconnection bus 514. The processor 512 can be any suitable processor, processing unit, or microprocessor, for example. Although not shown in FIG. 5, the system 510 can be a multi-processor system and, thus, can include one or more additional processors that are identical or similar to the processor 512 and that are communicatively coupled to the interconnection bus 514.

The processor 512 of FIG. 5 is coupled to a chipset 518, which includes a memory controller 520 and an input/output (“I/O”) controller 522. As is well known, a chipset typically provides I/O and memory management functions as well as a plurality of general purpose and/or special purpose registers, timers, etc. that are accessible or used by one or more processors coupled to the chipset 518. The memory controller 520 performs functions that enable the processor 512 (or processors if there are multiple processors) to access a system memory 524 and a mass storage memory 525.

The system memory 524 can include any desired type of volatile and/or non-volatile memory such as, for example, static random access memory (SRAM), dynamic random access memory (DRAM), flash memory, read-only memory (ROM), etc. The mass storage memory 525 can include any desired type of mass storage device including hard disk drives, optical drives, tape storage devices, etc.

The I/O controller 522 performs functions that enable the processor 512 to communicate with peripheral input/output (“I/O”) devices 526 and 528 and a network interface 530 via an I/O bus 532. The I/O devices 526 and 528 can be any desired type of I/O device such as, for example, a keyboard, a video display or monitor, a mouse, etc. The network interface 530 can be, for example, an Ethernet device, an asynchronous transfer mode (“ATM”) device, an 802.11 device, a DSL modem, a cable modem, a cellular modem, etc. that enables the processor system 510 to communicate with another processor system.

While the memory controller 520 and the I/O controller 522 are depicted in FIG. 5 as separate blocks within the chipset 518, the functions performed by these blocks can be integrated within a single semiconductor circuit or may be implemented using two or more separate integrated circuits.

Thus, certain examples provide for improved self-monitoring of medication intake by patients, especially, for example, elderly adults, resulting in fewer instances of under or overdosing. Additionally, certain examples provide for improved monitoring of patient medication intake by healthcare providers via a healthcare system, resulting in reduced stress on the healthcare providers. Furthermore, certain examples can be advantageously customized to analyze a variety of medications needed to treat a wide range of medical conditions.

Certain examples contemplate methods, systems and computer program products on any machine-readable media to implement functionality described above. Certain examples can be implemented using an existing computer processor, or by a special purpose computer processor incorporated for this or another purpose or by a hardwired and/or firmware system, for example.

One or more of the components of the systems and/or steps of the methods described above can be implemented alone or in combination in hardware, firmware, and/or as a set of instructions in software, for example. Certain examples can be provided as a set of instructions residing on a computer-readable medium, such as a memory, hard disk, DVD, or CD, for execution on a general purpose computer or other processing device. Certain examples of the present invention can omit one or more of the method steps and/or perform the steps in a different order than the order listed. For example, some steps cannot be performed in certain examples of the present invention. As a further example, certain steps can be performed in a different temporal order, including simultaneously, than listed above.

Certain examples include computer-readable media for carrying or having computer-executable instructions or data structures stored thereon. Such computer-readable media can be any available media that can be accessed by a general purpose or special purpose computer or other machine with a processor. By way of example, such computer-readable media can comprise RAM, ROM, PROM, EPROM, EEPROM, Flash, CD-ROM or other optical disk storage, magnetic disk storage or other magnetic storage devices, or any other medium which can be used to carry or store desired program code in the form of computer-executable instructions or data structures and which can be accessed by a general purpose or special purpose computer or other machine with a processor. Combinations of the above are also included within the scope of computer-readable media. Computer-executable instructions comprise, for example, instructions and data which cause a general purpose computer, special purpose computer, or special purpose processing machines to perform a certain function or group of functions.

Generally, computer-executable instructions include routines, programs, objects, components, data structures, etc., that perform particular tasks or implement particular abstract data types. Computer-executable instructions, associated data structures, and program modules represent examples of program code for executing steps of certain methods and systems disclosed herein. The particular sequence of such executable instructions or associated data structures represent examples of corresponding acts for implementing the functions described in such steps.

Embodiments of the present invention can be practiced in a networked environment using logical connections to one or more remote computers having processors. Logical connections can include a local area network (LAN) and a wide area network (WAN) that are presented here by way of example and not limitation. Such networking environments are commonplace in office-wide or enterprise-wide computer networks, intranets and the Internet and can use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention can also be practiced in distributed computing environments where tasks are performed by local and remote processing devices that are linked (either by hardwired links, wireless links, or by a combination of hardwired or wireless links) through a communications network. In a distributed computing environment, program modules can be located in both local and remote memory storage devices.

An exemplary system for implementing the overall system or portions of embodiments of the invention might include a general purpose computing device in the form of a computer, including a processing unit, a system memory, and a system bus that couples various system components including the system memory to the processing unit. The system memory can include read only memory (ROM) and random access memory (RAM). The computer can also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated computer-readable media provide nonvolatile storage of computer-executable instructions, data structures, program modules and other data for the computer.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method comprising:
obtaining a medication order including identification of a medication and a property associated with the medication, where the medication order is associated with a patient;
obtaining a value representing a characteristic of a physical state of the patient, where the value is measured by a sensor in contact with the patient;
determining whether the patient has taken the medication associated with the medication order based on a comparison of the value representing a characteristic of the physical state of the patient with the property associated with the medication; and
reporting whether the patient has taken the medication associated with the medication order via a user interface.

2. The method of claim 1, wherein the medication order has been uploaded to a healthcare system by a healthcare provider associated with the patient.

3. The method of claim 1, wherein the sensor comprises at least one of a sweat sensor or heart rate sensor.

4. The method of claim 1, wherein determining whether the patient has taken the medication associated with the medication order based on the value representing a characteristic of the physical state of the patient comprises comparing the collected value to a threshold.

5. The method of claim 4, wherein the threshold comprises a value representing a predicted effect of the medication on the characteristic of the physical state of the patient.

6. The method of claim 1, wherein the user interface comprises at least one of a visual display or audio feedback.

7. The method of claim 1, further comprising reporting whether the patient has taken the medication associated with the medication order to a healthcare system, where the information may be accessed by a healthcare provider.

8. A medication intake analysis and reporting system, said system comprising:
a medication analyzer to obtain a medication order including identification of a medication and a property associated with the medication, where the medication order is associated with a patient;
a patient analyzer to obtain a value representing a characteristic of a physical state of the patient, where the value is measured by a sensor in contact with the patient and to determine whether the patient has taken the medication associated with the medication order based on a comparison of the value representing a characteristic of the physical state of the patient with the property associated with the medication; and
a report generator to report whether the patient has taken the medication associated with the medication order.

9. The system of claim 8, further comprising a network interface to interface with a healthcare system to obtain the medication order.

10. The system of claim 9, wherein the medication order has been uploaded to the healthcare system by a healthcare provider associated with the patient.

11. The system of claim 8, wherein the sensor comprises at least one of a sweat sensor or heart rate sensor.

12. The system of claim 8, wherein the patient analyzer is to determine whether the patient has taken the medication associated with the medication order based on the value representing a characteristic of the physical state of the patient by comparing the collected value to a threshold.

13. The system of claim 11, wherein the threshold comprises a value representing a predicted affect of the medication on the characteristic of the physical state of the patient.

14. The system of claim 8, wherein the report generator is to report whether the patient has taken the medication associated with the medication order via a user interface.

15. The system of claim 13, wherein the user interface comprises at least one of a visual display or audio feedback.

16. The system of claim 8, wherein the report generator is to report whether the patient has taken the medication associated with the medication order to a healthcare system, where the information may be accessed by a healthcare provider.

17. A tangible computer readable medium having a set of instructions for execution on a processing device, the set of instructions implementing a method for medication intake analysis and review, said method comprising:
obtaining a medication order including identification of a medication and a property associated with the medication, where the medication order is associated with a patient;
obtaining a value representing a characteristic of a physical state of the patient, where the value is measured by a sensor in contact with the patient;
determining whether the patient has taken the medication associated with the medication order based on a comparison of the value representing a characteristic of the physical state of the patient with the property associated with the medication; and
reporting whether the patient has taken the medication associated with the medication order via a user interface.

18. The computer readable medium of claim 16, wherein the medication order has been uploaded to a healthcare system by a healthcare provider associated with the patient.

19. The computer readable medium of claim 16, wherein the sensor comprises at least one of a sweat sensor or heart rate sensor.

20. The computer readable medium of claim 16, wherein determining whether the patient has taken the medication associated with the medication order based on the value representing a characteristic of the physical state of the patient comprises comparing the collected value to a threshold.

21. The computer readable medium of claim 19, wherein the threshold comprises a value representing a predicted affect of the medication on the characteristic of the physical state of the patient.

22. The computer readable medium of claim 16, wherein the user interface comprises at least one of a visual display or audio feedback.

23. The computer readable medium of claim 16, further comprising reporting whether the patient has taken the medication associated with the medication order to a healthcare system, where the information may be accessed by a healthcare provider.
